(19) United States
(12) Patent Application Publication
(10) Pub. No.: US 2004/0215305 A1
(43) Pub. Date: Oct. 28, 2004

(54) Implantable lead with intermediate insertion port for receiving a stiffening member

(76) Inventor: Shahn S. Sage, Andover, MN (US)

Correspondence Address: INTELLECTUAL PROPERTY GROUP, FREDRIKSON & BYRON, P.A., 200 SOUTH SIXTH STREET, SUITE 4000, MINNEAPOLIS, MN, 55402, US

(21) Appl. No.: 10/423,179

(22) Filed: Apr. 25, 2003

(51) Int. Cl. A61N 1/05

(52) U.S. Cl. 607/122

(57) ABSTRACT

Implantable leads, lead assemblies, and methods for implanting the leads into the human body. The lead can include numerous distal electrical contacts for sensing and/or stimulation as well as numerous proximal contacts for connecting to a lead extension and/or an implantable pulse generator. The lead includes a stylet lumen extending between a side-wall stylet entrance port and the lead distal region. Some leads further include a stylet guide disposed near the stylet opening for fluoroscopic visualization and/or insuring proper insertion and distal advancement of the stylet within the lead. In use, the stylet can be inserted through the lead stylet port to stiffen only the distal portion of the lead, and the distal portion of the lead inserted through an introducer needle into the human body. The stiffened distal portion of the lead can be short and easily controllable. Implantable leads having an intermediate stylet insertion port find one use as neurological stimulation leads for implantation within the intrathecal spaces of the spinal column to manage pain. The short, stiffened portion of lead allows for a longer total lead length which can eliminate the need for lead extensions in some applications.

### FIELD OF THE INVENTION

The present invention is related generally to medical devices. More specifically, the present invention is related to implantable, electrical neurological leads.

### BACKGROUND OF THE INVENTION

Implantable leads having externally exposed ring or band electrodes can be used to both deliver electrical stimulation to surrounding tissue and to sense electrical energy produced by the surrounding tissue. Such leads are often implanted within the epidural or intrathecal spaces of the spinal column, along peripheral nerves, within the brain, and about the heart. Electrical stimulation in the spinal cord has been shown to be effective in relieving intractable pain in some patients. Such electrical stimulation can reduce or eliminate the use of pain relieving drugs.

A neurological stimulation lead is commonly used to deliver the electrical signals. One such lead is formed of polymeric material, for example, polyurethane or silicone. The lead can be nominally 1 mm in outer diameter and about 20 cm in length. A typical lead may have a series of electrodes formed as bands or rings disposed in a spaced apart relationship in a lead distal region. The distal region of the lead can later be introduced into the spinal column. One such lead has eight electrodes in the distal region, with each electrode having its own conductor extending the length of the lead to a proximal lead region. The lead proximal region can have a corresponding set of band or ring connectors or terminals, one for each corresponding electrode in the distal region. Each proximal region terminal can thus be connected to one distal electrode in a typical configuration.

The terminals can be used to couple the proximal end of the lead to a lead extension which can in turn be coupled to an implantable pulse generator (IPG). The lead extension can provide added length to extend the reach of the lead to a more distantly placed IPG. In some embodiments, the lead extension is between about 20 and 50 cm in length.

The lead typically has a lumen extending from the proximal end through to the distal region, with the lumen being dimensioned to accept a stiffening member or stylet. The lead, commonly formed of a polymeric material and being very small in cross section, is typically very floppy and not pushable. With a stylet or stiffening member inserted, the lead gains the needed pushability, and can be advanced into and up the spinal column to the desired location.

In use, a large gauge Toughy needle can be inserted into the spinal column and into the spinal canal. The stylet is then inserted into the lead, and the now stiffened lead advanced through the needle and up into the spinal canal. When the distal region of the lead is in the proper position, the stiffening member can be removed and the introducing needle also removed, leaving the proximal end of the lead protruding from the patient.

A small incision can then be made near the site of entry of the lead, in order to direct the proximal end of the neurological lead back into the body to be mated to a lead extension or to the IPG. The proximal end of the lead extension is coupled to the proximal end of the lead, and electrical continuity established. The lead extension is used to extend the useful length of the lead sufficient to reach the implanted IPG, which can be, for example, 20-50 cm distant. With the length increased by the extension, the free end of the extension can be inserted into the incision and into the body.

In one procedure, known as “tunneling”, an elongate, flexible metal device is used to form a tunnel or passageway under the skin, for example, around the torso, to the site of the implanted or soon to be implanted IPG. This tunneling procedure can be used to form the passageway for the extension which is then advanced through the passageway and to the IPG site.

The extension, while adding length, also adds complexity and cost. It also adds yet another required step for the treating physician to perform. Increasing the lead length to do away with the need for the extension would be desirable. However, with current leads, the stiffening member or stylet would likewise have to be increased in length. While this is possible, a significantly longer lead having a stiffening member within would be somewhat unwieldy. For example, a 50 cm long stylet inserted within a 50 cm long lead would be difficult for the treating physician to maneuver. This aspect is significantly limiting, given that the treatment site is the spinal cord, where care must be exercised.

What would be desirable are neurological stimulating leads which are sufficiently long so as to be able to do away with any required extension. What would be advantageous are long neurological leads providing distal stiffness and pushability without being unwieldy for the treating physician. What would also be beneficial are neurological leads which can be provided in a variety of lengths all sharing a common stiffening member, not requiring a different stiffening member to match each size lead.

### SUMMARY OF THE INVENTION

The present invention provides an implantable lead including a proximal region, a distal region, an intermediate region disposed between the proximal and distal regions, and a side wall. The lead further includes at least one proximal contact and at least one distal contact, electrically coupled to each other by a conductor. The lead includes a lumen disposed through the lead body between the distal region and the intermediate region and a stylet port providing access between the lead body sidewall and the lumen in the intermediate region. The stylet port and lumen can be adapted to slidably receive a stiffening member or stylet through the port and into the lead distal region. Some embodiments of the present invention include an implantable lead assembly including the implantable lead and a stylet adapted to be slidably received in the implantable lead stylet port and stylet lumen.

Some implantable leads have a plurality of distal contacts and a plurality of proximal contacts, with the stylet port disposed between the innermost of the distal and the proximal contacts. Some leads have a radiopaque marker disposed nearer the stylet port for locating the stylet port under fluoroscopy. The marker can include a radiopaque seal disposed at least partially in the stylet port. Some leads include a stylet guide disposed about the stylet port, the stylet guide including an aperture for receiving the stylet. Some stylet guides include a stylet guide arm extending radially and distally into the stylet port for distally directing an inserted stylet. The stylet guide can be formed of, and/or plated with, a radiopaque material such as gold or platinum.

In use, an implantable lead having the desired total length and suitable characteristics is selected for implantation. The lead preferably has a stylet lumen extending between the lead side wall stylet port and the lead distal region. The length between the stylet port and the end of the stylet lumen is preferably dimensioned to receive the stylet, regardless of the total lead length. An introducer needle can be advanced into the body near the implantation site. The stiffening member or stylet can be inserted into the lead stylet port and further distally into the lead to stiffen the lead distal region. The stiffened lead can be inserted through the lumen of the introducer needle and further distally from the needle to the target site. The stylet can be retracted from the lead, and the introducer needle retracted as well, leaving the lead proximal region extending from the body. In some methods, the lead proximal end is coupled to a lead extension before being tunneled into the body to fully implant the lead.

The lead, lead assembly, and lead implantation methods allow for implanting a variety of lead lengths while stiffening only the lead distal portion. The lead portion disposed proximal of the stylet port need not be stiffened and/or include a stylet lumen within. This remaining lead proximal portion can be sufficiently long to eliminate the need for a lead extension by not requiring that the entire lead length accept a stylet to provide for stiffening. A single length stylet may thus be used with a family of leads, each having a different total length, but having a similar length stylet lumen within.

### DESCRIPTION OF THE DRAWINGS

FIG. 1 is a side view of an implantable lead having an intermediate stylet insertion port and a stylet for insertion into the port;

FIG. 2 is a fragmentary, longitudinal cross-sectional view of a distal portion of the lead of FIG. 1;

FIG. 3 is a transverse, cross-sectional view taken through 3-3 of FIG. 2, showing a distal tri-lumen portion of the lead;

FIG. 4 is a perspective view of a stylet guide or marker which can be positioned over the intermediate insertion port of a lead;

FIG. 5 is a fragmentary, cutaway view of a lead intermediate portion having the stylet marker of FIG. 4 guiding a stylet into the intermediate insertion port;

FIG. 6 is a fragmentary, longitudinal, cross-sectional view of the lead intermediate portion of FIG. 5, having a sealing filler material sealing the intermediate insertion port;

FIG. 7 is a fragmentary, perspective view of an assembly having a stylet handle adapted to grip a neurological lead; and

FIG. 8 is a transverse, cross-sectional view of the handle of FIG. 7, having the lead held within the longitudinal channel.

### DETAILED DESCRIPTION OF THE PREFERRED EMBODIMENTS

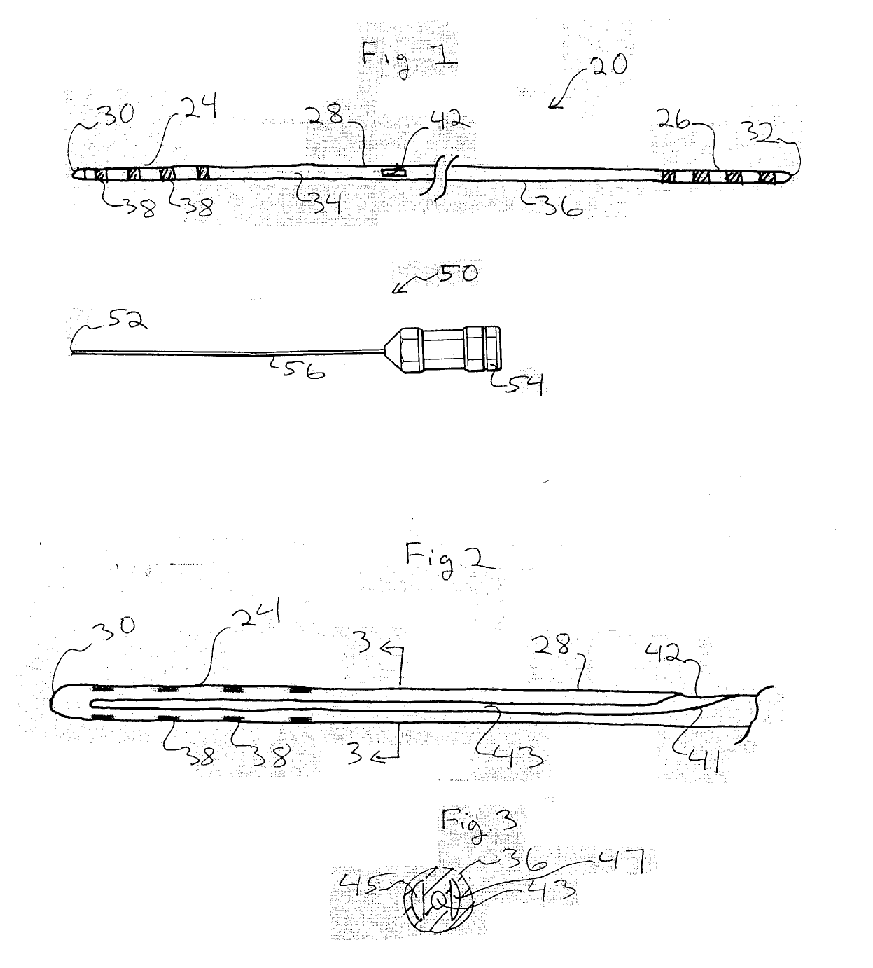

FIG. 1 illustrates a neurological stimulation lead 20 having a distal region 24, a proximal region 26, and an immediate region 28 disposed between the distal and proximal regions. In a preferred embodiment, the intermediate region is defined to lie between the innermost distal and proximal electrical contacts described below. A stylet entrance or insertion part 42 may be seen in intermediate region 28. Lead 20 can be formed of a body or shaft 34 extending between a distal end 30 and a proximal end 32. Lead body 34 has an exterior surface or tubular side wall 36. Lead body 34 is preferably formed of a polymeric material, for example, polyurethane or silicone.

Lead distal region 24 can include a number of electrodes 38 which can be disposed concentrically about lead body 34 in a spaced-apart configuration. Electrodes 38 may also be described as electrical contacts or contacts. Electrodes 38 are normally adapted to be inserted into the human body, are externally exposed, and can be used for neurological stimulation. One exemplary use of electrodes 38 is the stimulation of the nerves within the spinal cord. Proximal region 26 can include a number of connector bands or connector rings 40 disposed in a spaced-apart configuration. Connectors 40 may also be described as electrical contacts or terminals, and are preferably also externally exposed. Electrodes 38 and connectors 40 may be formed of Platinum and/or Iridium. Connectors 40 can be used for connecting lead 20 to a lead extension to extend the effective length of the lead. In some uses, connectors 40 may also be used to directly couple lead 22 to an implantable pulse generator.

Electrodes 38 and connectors 40 can be coupled to each other in a one-to-one arrangement. In some leads, the distal-most electrode is coupled to the distal-most connector, the second-to-distal-most electrode coupled to the second-to-distal-most connector, and so forth. The electrodes and connectors can be coupled through conductors extending between the two. In some leads, the conductors are embedded within the lead while in other leads, the conductors lie within lumens extending the length of the lead. In some leads, the conductors are disposed within lumens which are later backfilled to substantially fill the lumens with a polymeric material.

FIG. 1 also illustrates a stylet 50. Stylet 50 includes generally a shaft 56 extending between a distal tip 52 and a proximal end or handle 54. Stylet 50 is typically dimensioned to be slideably received within stylet entrance 42 and further within a lumen extending distally from stylet entrance 42 toward distal region 24.

Lead 20 can be varied in outer diameter and length to suit the application for which it is intended. In some embodiments, lead 20 has a total length of between about 5 cm and about 100 cm. In other embodiments, lead 20 has an outer diameter of less than about 1 mm and a total length of between about 10 cm and 150 cm. The lead length between stylet entrance 42 and distal end 30 can vary as well. In some embodiments, the distance from stylet entrance 42 to distal end 30 is less than 50 cm, preferably less than 30 cm, and most preferably less than about 20 cm. Stylet 50 preferably has a length adapted to approximately match the length between stylet entrance 42 and distal end 30. Stylet 50 preferably has a shaft outer diameter of less than about 0.050 inches, more preferably less than about 0.020 inches, and most preferably less than about 0.010 inches.

FIG. 2 illustrates a distal portion of lead 20 in longitudinal cross-section. Lead 20 may be seen to have a flared lumen portion 41 extending from stylet entrance or port 42 toward distal region 24. A stylet lumen 43 may be seen to extend distally from stylet opening 42. In the embodiment illustrated, stylet lumen 43 terminates proximal of lead distal end 30. In some leads, port 42 lies at an angle of between about 20 and 60 degrees from the longitudinal axis of the lead. In one lead embodiment, port 42 lies at an angle of about 30 degrees from the lead longitudinal axis. In other embodiments, port 42 is dimensioned and configured such that a stiffening member inserted into lead 20 to distal region 24 lies at an angle of less than about 40 degrees, preferably about 30 degrees, from the lead longitudinal axis near port 42. Leads preferably have a distal region wall thickness of at least about 0.004 inch. In some leads, a plug material can be disposed within or about stylet port 42 in order to provide a seal between the lead lumen or lumens and the body in which it is inserted. In some leads, a silicone polymeric material carrying a radiopaque marker material is used to plug stylet port 42. In one such plug material, silicone carrying a barium oxide radiopaque material is used as the plug material. The plug or seal material can be inserted into port 42 during manufacture. The stylet distal tip can be advanced through the silicone sealing material to stiffen the lead, and can later be retracted. In some embodiments, radiopaque marker bands can be disposed near one or both sides of stylet port 42 in order to better mark the stylet port for identification of the stylet opening location under fluoroscopy, should surgical access to the port later be required.

FIG. 3 illustrates a transverse cross-section taken through a distal portion of lead 20. Lead 20 may be seen to have a tri-lumen configuration in FIG. 3, having a first lumen 45, a second lumen 47, and a third or stylet lumen 43. The tri-lumen configuration can be used to provide lumens for conductors extending between the electrodes and connectors, while separating the conductors from the inserted stylet. The conductors may lie within first and second lumens 45 and 47, leaving lumen 43 clear to receive a stylet. In some leads, a mono-lumen configuration may be found proximal of the stylet opening, where the stylet presence need not be adapted for, leaving a larger cross-sectional area for the conductors.

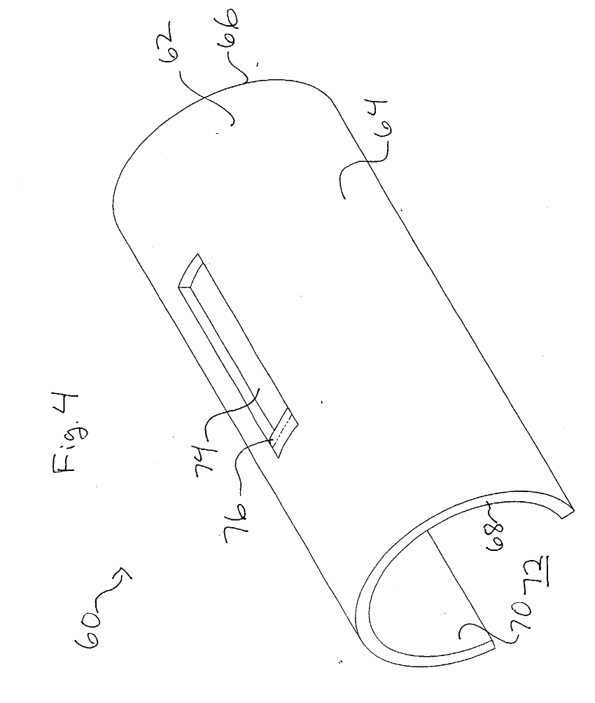

FIG. 4 illustrates a stylet guide or marker 60 for augmenting and marking stylet entrance 42. Stylet guide 60 can be used to both mark the stylet entrance and to aid in proper insertion of the stylet into the lead. Stylet guide 60 may be seen to have generally a distal end 66, a proximal end 68, a substantially cylindrical body 62, an outer surface 64, and an inner surface 70. Cylindrical body 62 may be seen to include a gap 72, an aperture 74, and a deflector arm 76 for properly directing a stylet into the stylet lumen. Stylet guide 60 may be made from sheet metal, for example, stainless steel. The sheet metal may be stamped into the appropriate shape, with aperture 74 being formed through body 62 and deflector arm 76 pushed downward from aperture 74. Stylet guide 60 may then be plated with a radiopaque marker material, for example, gold or platinum.

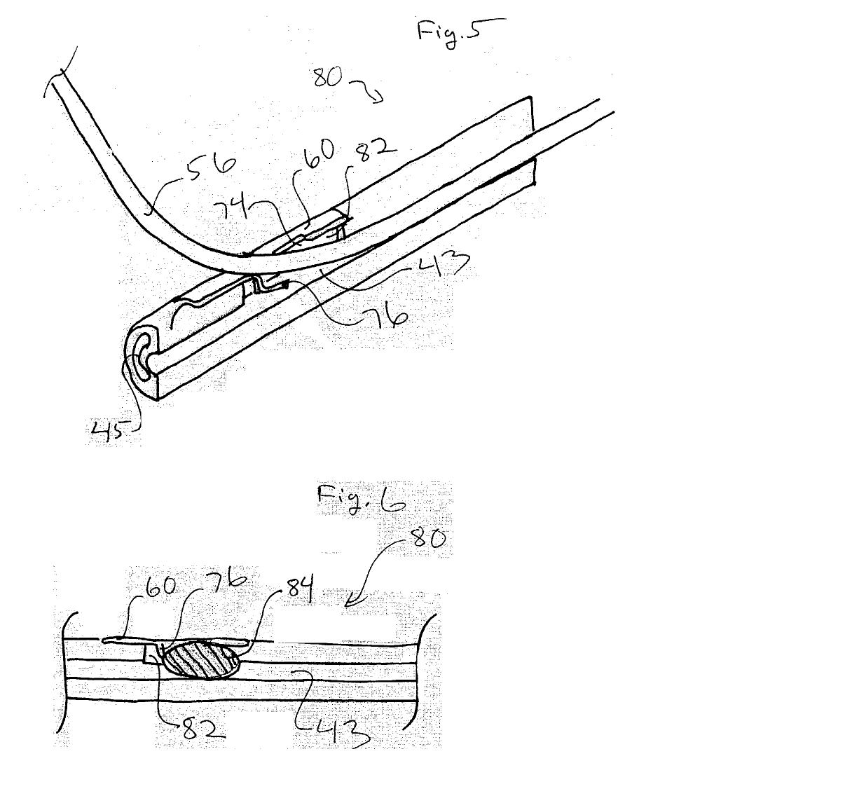

FIG. 5 illustrates an intermediate portion of another neurological lead 80, similar in many respects to lead 20 of FIG. 1, and sharing some identically numbered features. Lead 80 differs in having a stylet port or opening 82 which is not flared. Lead 80 includes stylet guide or marker 60 of FIG. 4 disposed over and about stylet opening 82. Stylet deflector arm 76 may be seen guiding stylet shaft 56 distally into stylet lumen 43. First conductor lumen 45 may also be seen, previously discussed with respect to FIG. 3. As may be seen from inspection of FIG. 5, deflector arm 76 extends radially inward and distally along stylet guide aperture 74 to ensure that stylet shaft 56 can be inserted in only the distal direction. Stylet guide 60 can also serve to provide a large radiopaque marker easily visible under fluoroscopy.

FIG. 6 further illustrates lead 80, having a seal or filler material plug 84 disposed within stylet opening 82. Stylet guide 60 and deflector arm 76 may also be seen in FIG. 6. In some methods, seal 84 is injected or placed after withdrawal of the stylet and proper placement of the lead. In a preferred embodiment, seal 84 is injected or otherwise placed within stylet opening 82 during manufacture, with the stylet being inserted through the soft pliable material.

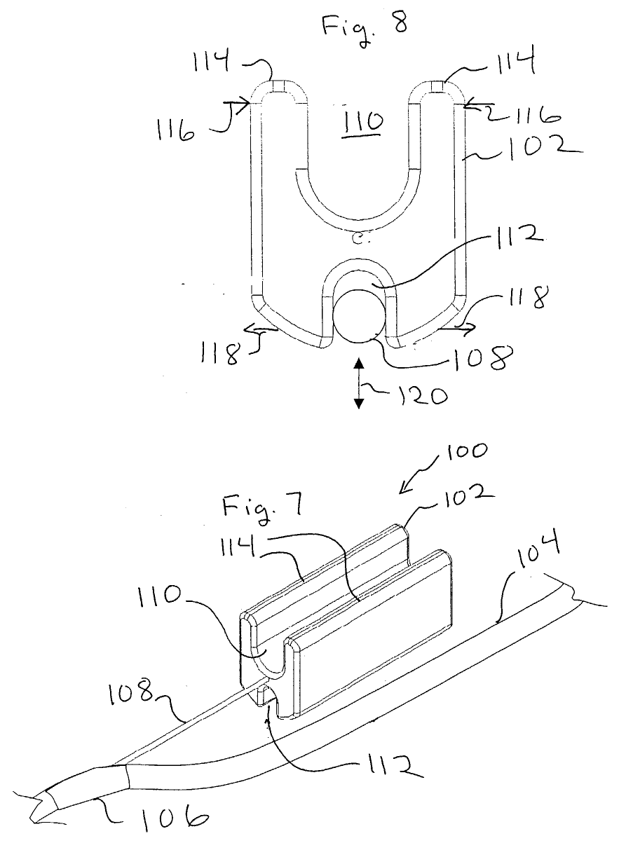

FIG. 7 illustrates an assembly 100 including a stylet wire or shaft 108 coupled to a stylet proximal handle 102 being inserted into a neurological lead 104. Stylet wire 108 is in the process of being advanced into a stylet port (not visible in FIG. 7) in an intermediate portion 106 of lead 104. Stylet handle 102 includes a first, longitudinal channel 112 and a second, longitudinal channel 110. Second longitudinal channel 110 may be seen to be disposed within handles or wings 114. In some embodiments, the first longitudinal channel 112 is dimensioned to firmly grasp and hold lead 104 within.

FIG. 8 illustrates stylet handle 102 having stylet wire 108 held within first longitudinal channel 112. In some embodiments, wings 114 can be squeezed together, as indicated at 116. This squeezing motion can force apart the portions of handle 102 on either side of first longitudinal channel 112, as indicated at 118, thereby increasing the width of first longitudinal channel 112. With the width slightly increased, stylet wire 108 can be more easily transversely forced into first longitudinal channel 112, as indicated at 120. In other embodiments, handle 102 is formed of sufficiently elastic material to allow stylet wire 108 to be forced into, and retrieved from, first channel 112 without squeezing on the opposite side of the handle. In still another embodiment, the stylet handle is formed from a substantially round cross-section, cylindrical, elongate material, having the first, and second channels formed on opposite sides of the elongated cylinder.

Handle 102 can be formed of a polymeric material, for example, a thermoset plastic. In one embodiment, first longitudinal channel 112 has about 0.050 inch width and about a 0.07 to 0.08 inch depth. Second longitudinal channel 110 can have a width of about 0.2 inches and a depth of about 0.25 inches. The handle can have a nominal width of about 3/5ths inch and a length of 0.5 inch. In some embodiments, the handle is formed from a 3/5ths inch outer diameter cylinder, and the channels formed into opposite surfaces of the cylindrical piece.

In use, stylet wire 108 can be advanced into the insertion port in lead 104 until the stylet wire is substantially totally advanced into the lead. The lead can then be transversely forced into the gripping first longitudinal channel, for example, by squeezing opposing wings on the handle. The handle and the lead are now aligned and move as a single unit. In particular, the handle and lead now rotate together. The combined stylet and lead can now be advanced into the introducer needle, as described below with respect to the invention generally.

In use, an introducer needle, for example, a 14 or 16 gauge Toughy needle, may be advanced into the intrathecal space in the spinal column. The stylet may then be advanced distally through the stylet opening to stiffen the lead. The now stiffened lead may be advanced distally through the positioned introducer needle and into the intrathecal space in the spinal column. The lead can be advanced upward through the spinal canal, past the distal tip of the introducer needle. When the lead has been properly positioned, the stylet can be retracted from the lead, and the introducer needle retracted from about the lead. The proximal region of the lead, extending from the body, can be properly coupled to a lead extension or directly to an implantable pulse generator. The free end of the lead or lead extension can also be “tunneled” to an appropriate site within the body for appropriate coupling to an implantable pulse generator.

Referring again to FIG. 1, other aspects of the invention may be further discussed. Inspection of FIG. 1 shows a length between stylet opening 42 and distal end 30 as well as a length between stylet opening 42 and proximal end 32. Different applications and different target sites call for a different total length for lead 20 between distal end 30 and proximal end 32. Different treatments and target sites may also differ as to the length of lead 20 to be inserted into the body.

In previous leads, leads having a different total length required stylets or stiffening members having a corresponding different length to match the lead length. The present invention provides for a series of leads, each having a different total length, but allowing for use of the same length stylet, provided that the lead length between the stylet entrance and the distal end of the stylet lumen is adapted to receive the same stylet. In one example of the invention, a stylet having a length of about 15 cm can be used with each member of a family of varying length leads having a distance from stylet entrance to distal end of about 15 cm. This allows the treating physician to select the appropriate lead while using the same stylet. As previously discussed, the present invention also provides a design which can eliminate the need for a lead extension, as the proximal portion can be quite long.

The present invention can provide improved neurological stimulation leads. This exemplary use of the present invention is not limiting however. The present invention explicitly includes implantable leads for both stimulation and sensing, and for implantation in non-spinal sites, for example, brain and cardiac implantation sites.

The foregoing detailed description should be read with reference to the drawings, in which like elements in different drawings are numbered identically. The drawings, which are not necessarily to scale, depict selected embodiments and are not intended to limit the scope of the invention. Several forms of invention have been shown and described, and other forms will now be apparent to those skilled in art. It will be understood that embodiments shown in drawings and described above are merely for illustrative purposes, and are not intended to limit scope of the invention as defined in the claims which follow.

What is claimed is:

1. An implantable lead comprising:
an elongate lead body comprising a proximal region, a distal region, an intermediate region disposed between the proximal and distal regions, and a sidewall;
at least one conductor disposed within the lead body and extending from the proximal region to the distal region;
at least one proximal contact disposed in the lead body proximal region and in electrical contact with the at least one conductor;
at least one distal contact disposed in the lead body distal region and in electrical contact with the at least one conductor;
a lumen disposed through the lead body between the distal region and the intermediate region; and
a port providing access through the lead body sidewall to the lumen in the intermediate region.

2. An implantable lead as in claim 1, wherein the at least one proximal contact comprises a plurality of proximal contacts and wherein the at least one distal contact comprises a plurality of distal contacts, wherein the port is disposed between the innermost of the proximal and distal contacts.

3. An implantable lead as in claim 1, further comprising a radiopaque marker disposed near the port.

4. An implantable lead as in claim 3, wherein the marker includes a radiopaque seal disposed at least partially in the port.

5. An implantable lead as in claim 1, further comprising a stylet guide disposed about the port, the stylet guide including an aperture therethrough for receiving the stylet.

6. An implantable lead as in claim 5, wherein the stylet guide includes a stylet guide arm extending radially and distally into the port for distally directing an inserted stylet.

7. An implantable lead as in claim 1, wherein the lead has an outer diameter of less than about 1 millimeter and a total length of between about 10 cm. and about 150 cm.

8. An implantable lead as in claim 1, wherein the lead has a longitudinal axis and wherein the port is dimensioned and configured such that a stiffening member inserted into the lead from the intermediate region to the distal region lies at an angle of less than about 40 degrees from the lead longitudinal axis near the port.

9. An implantable lead comprising:
an elongate lead body comprising a proximal region, a distal region, and an intermediate region disposed between the proximal and distal regions;
means for providing electrical continuity between the proximal and distal regions; and
means for reversibly stiffening the lead between the intermediate and distal regions while not stiffening the lead between the intermediate and proximal regions.

10. An implantable lead as in claim 9, wherein the means for reversibly stiffening the lead comprises means for receiving a stiffening member into the lead body for disposition between the intermediate region and the distal region.

11. An implantable lead as in claim 10, wherein the means for providing electrical continuity comprises a plurality of proximal electrical contacts electrically coupled to a plurality of distal electrical contacts, wherein the means for receiving a stiffening member is disposed between the innermost of the proximal and distal contacts.

12. An implantable lead as in claim 9, further comprising means for radiopaquely marking the lead intermediate region

13. An implantable lead as in claim 10, further comprising means for radiopaquely marking and sealing the means for receiving the stiffening member.

14. An implantable lead as in claim 10, wherein the means for receiving a stiffening member further comprises means for guiding the stiffening member into the lead body.

15. An implantable lead as in claim 14, wherein the means for guiding the stiffening member into the lead body includes means for distally directing the stiffening member into the lead body.

16. An implantable lead as in claim 9, wherein the lead has an outer diameter of less than about 1 millimeter and a total length of between about 10 cm. and about 150 cm.

17. An implantable lead assembly comprising:
an elongate stiffening member having a proximal region and a distal region;
an elongate lead body comprising a proximal region, a distal region, an intermediate region disposed between the proximal and distal regions, and a sidewall;
at least one conductor disposed within the lead body and extending from the lead body proximal region to the lead body distal region;
at least one proximal contact disposed in the lead body proximal region and in electrical contact with the at least one conductor;
at least one distal contact disposed in the lead body distal region and in electrical contact with the at least one conductor;
a lumen disposed through the lead body between the lead body distal region and the lead body intermediate region; and
a port providing access between the lead body sidewall and the lumen in the lead body intermediate region, wherein the port and lumen are dimensioned to slidably receive the elongate stiffening member.

18. An implantable lead assembly as in claim 17, wherein the stiffening member further comprises a handle secured to the proximal region, wherein the handle includes a channel dimensioned to receive and releasable grip the lead body.

19. A method for inserting an implantable lead into the human body, the method comprising:
providing an elongate stiffening member having a proximal region and a distal region;
providing an elongate lead body comprising: a proximal region, a distal region, an intermediate region disposed between the proximal and distal regions, and a sidewall; at least one conductor disposed within the lead body and extending from the lead body proximal region to the lead body distal region; at least one proximal contact disposed in the lead body proximal region and in electrical contact with the at least one conductor; at least one distal contact disposed in the lead body distal region and in electrical contact with the at least one conductor; a lumen disposed through the lead body between the lead body distal region and the lead body intermediate region; and a port providing access between the lead body sidewall and the lumen in the lead body intermediate region, wherein the port and lumen are dimensioned to slidably receive the elongate stiffening member; and
inserting the lead distal region into the human body while carrying the stiffening member at least partially within the lumen between the port and the lead distal region.

20. A method for inserting an implantable lead into the human body as in claim 19, further comprising inserting the stiffening member into the lead through the lead port before inserting the lead distal region into the human body.

21. A method for inserting an implantable lead into the human body as in claim 20, further comprising providing an introducer needle and inserting the introducer needle into the human body, wherein the inserting implantable lead step includes inserting the lead carrying the stiffening member into the introducer needle.

22. A method for inserting an implantable lead into the human body as in claim 19, further comprising retracting the stiffening member from the inserted lead distal region.
